(19) United States
(12) Patent Application Publication
(10) Pub. No.: US 2024/0277354 A1
(43) Pub. Date: Aug. 22, 2024

(54) ASPIRATION CATHETER WITH STABILIZING STRUCTURAL MEMBER MINIMIZING RISK OF DAMAGE TO AN INNER WALL OF A VESSEL DURING ASPIRATION

(71) Applicant: Neuravi Limited, Galway (IE)

(72) Inventors: Karl KEATING, Galway (IE); AnnaLisa SMULLIN, Galway (IE); Aoife GLYNN, Galway (IE); Sarah JOHNSON, Galway (IE); David QUINN, Galway (IE); Peter MONAGHAN, Galway (IE); Artur SPYCHALA, Galway (IE); Petrica STEFANOV, Loughrea (IE); Avril O’HIGGINS, Galway (IE); Jane EGWARI, Galway (IE); Colm PYNE, Galway (IE); David VALE, Barna (IE); Patrick GRIFFIN, Castlegar (IE)

(73) Assignee: Neuravi Limited, Galway (IE)

(21) Appl. No.: 18/441,265

(22) Filed: Feb. 14, 2024

Related U.S. Application Data
(60) Provisional application No. 63/447,506, filed on Feb. 22, 2023.

(51) Int. Cl. A61B 17/22; A61B 17/00; A61M 25/00; A61M 25/01; A61M 25/10

(52) U.S. Cl. CPC A61B 17/22; A61B 2017/00862; A61B 2017/00867; A61B 2017/22067; A61B 2017/22079; A61M 2025/0059; A61M 25/0074; A61M 25/0158; A61M 2025/1052

(57) ABSTRACT

An aspiration catheter including a body having a proximal end and an opposite distal tip with a lumen extending in a longitudinal direction therethrough from the proximal end to the distal tip. The aspiration catheter further includes a stabilizing structural member stabilizing the body relative to the inner wall of the vessel to minimize movement and/or radial expansion of the distal tip of the body while subject to aspiration that otherwise may potentially damage the inner wall of the vessel.

### CROSS-REFERENCE TO RELATED APPLICATION

This application claims the benefit of U.S. Provisional Patent Application No. 63/447,506, filed on Feb. 22, 2023, the disclosure of which is incorporated herein by reference in its entirety.

### FIELD

The present disclosure generally relates to an aspiration catheter for use during a thrombectomy procedure to capture and remove a targeted clot. Preferably the present disclosure relates to an improved aspiration catheter subject to aspiration (e.g., vacuum (i.e., non-cyclic) or pulsatile/cyclic), wherein the aspiration catheter is configured to minimize potential risk of damage to the inner wall of the vessel wall by stabilizing the body relative to the inner wall of the vessel to minimize movement and/or radial overexpansion of the distal tip.

### BACKGROUND

During endovascular treatment to capture and remove a clot, aspiration (e.g., vacuum or pulsatile/cyclic) is often applied to a catheter to improve efficiency. However, when subject to aspiration (e.g., vacuum or pulsatile/cyclic), undesirable excessive movement imparted to the distal tip/end of the aspiration catheter poses a significant risk of potential damage to the inner wall of the vessel. It is therefore desirable to develop an improved aspiration catheter that provides greater stability during aspiration thereby diminishing or reducing the risk of potential damage to the inner wall of the vessel.

Also, when using aspiration (e.g., vacuum or pulsatile/cyclic) to capture and remove a clot the aspiration catheter may be configured to have a radially enlargeable distal section (e.g., funnel distal section) to aid in ingestion of the clot into the lumen of the body or shaft. However, if the diameter of the clot being ingested exceeds that of the enlarged distal end in a radially expanded or deployed state within the vessel the funnel distal section or the aspiration catheter will over expand radially wider posing a significant risk of damaging the inner wall of the vessel and/or becoming stuck. It is therefore desirable to develop an improved aspiration catheter that prohibits overexpansion of the radially enlargeable distal section (e.g., funnel distal section).

In addressing these issues it is desirable to develop an improved aspiration catheter that reduces the risk of damage to the inner wall of the vessel during aspiration.

### SUMMARY

An aspect of the present disclosure is directed to an improved aspiration catheter minimizing risk of damage to the inner wall of the vessel during aspiration (e.g., vacuum or pulsatile/cyclic).

An aspect of the present disclosure is directed to an improved aspiration catheter to efficiently capture the target clot therein while providing greater stability against excessive movement of the distal tip/end of the aspiration catheter thereby minimizing risk of damage to the inner wall of the vessel during aspiration (e.g., vacuum or pulsatile/cyclic).

A further aspect of the present disclosure is directed to an improved aspiration catheter stabilized via a stabilizing structural member thereby minimizing risk of damage to the inner wall of the vessel resulting from excessive movement of the distal/tip during aspiration (e.g., vacuum or pulsatile/cyclic).

A still further aspect of the present disclosure is directed to an improved aspiration catheter including a stabilizing structural member to prevent overexpansion of an enlargeable distal section (e.g., funnel distal section) thereby minimizing risk of damage to the inner wall of the vessel and/or risk of the clot becoming stuck therein during aspiration (e.g., vacuum or pulsatile/cyclic).

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices, by way of example only, not by way of limitation.

FIG. 1A depicts a distal section of an example aspiration catheter with a stabilizing structural member (e.g., tether) for preventing overexpansion of a radially enlargeable distal section in accordance with the present disclosure; wherein the aspiration catheter is depicted while being delivered through the vessel to the target site with the radially enlargeable distal section while sheathed within an outer guide;

FIG. 1B depicts the radially enlarged distal section of the aspiration catheter unsheathed from the outer guide, wherein overexpansion of the radially enlargeable distal section resulting from ingestion of the oversized clot therein is prohibited by the tether;

FIG. 2A depicts a distal section of another example of an aspiration catheter having an axially compressible bellows section as a stabilizing structural member in accordance with the present disclosure, wherein the axially compressible bellows section is illustrated in a non-stabilizing (i.e., axially non-compressed) state;

FIG. 2B depicts the distal section of the example aspiration catheter of FIG. 2A, wherein the axially compressible bellows section is illustrated in a stabilizing state (i.e., axially compressed) state;

FIG. 3A depicts the distal section of still another example aspiration catheter having an external inflatable balloon sleeve as a stabilizing structural member in accordance with the present disclosure, wherein the external inflatable balloon sleeve is illustrated in a non-stabilizing (i.e., non-inflated) state;

FIG. 3B depicts the distal section of the example aspiration catheter of FIG. 3A, wherein the external inflatable balloon sleeve is illustrated in a stabilizing (i.e., inflated) state;

FIG. 4A depicts the distal section of yet another example of an aspiration catheter including a pulling member extending axially through the lumen and secured to the distal section, wherein the pulling member is illustrated in a non-deployed or non-actuated state while the aspiration catheter is in a non-stabilizing state;

FIG. 4B depicts the distal section of the example aspiration catheter of FIG. 4A, wherein the pulling member is illustrated in a deployed or actuated state altering (e.g., buckling, snaking, or spiraling) the axial profile of the aspiration catheter to a stabilizing state;

FIG. 5A depicts the distal section of another example of an aspiration catheter having an external pre-filled gel sleeve as a stabilizing structural member in accordance with the present disclosure and actuated (i.e., axially compressed) via a pulling member extending in an axial direction through the lumen and secured to the distal section of the aspiration catheter, wherein the external pre-filled gel sleeve is illustrated in a non-stabilizing (i.e., axially non-compressed) state prior to pulling on the pulling member;

FIG. 5B depicts the distal section of the example aspiration catheter of FIG. 3A, wherein the external pre-filled gel sleeve is illustrated in a stabilizing (i.e., axially compressed) state in response to pulling on the pulling member;

FIG. 6A depicts the distal section of still another example of an aspiration catheter including a nitinol wire acting as the stabilizing structural member is thermally activatable by an electrical circuit; wherein the nitinol wire is illustrated in a non-stabilizing state (i.e., Martensite phase—temperature above the Austenite transformation finish temperature) so that distal section of the aspiration catheter remains compliant and trackable;

FIG. 6B depicts the distal section of the example aspiration catheter of FIG. 6B at the target site during aspiration wherein the nitinol wire is illustrated in a stabilizing state (i.e., Austenite Phase—temperature below the Austenite transformation temperature) stiffening the distal section of the aspiration catheter to stabilize the distal tip/end;

FIG. 7 is modification of FIG. 6A of an example aspiration catheter at the target site during aspiration the nitinol wire set herein to a curved, spiral, or undulating shape while in a stabilizing state (i.e., Austenite Phase—temperature below the Austenite transformation temperature) stiffening the distal section of the aspiration catheter to stabilize the body relative to the inner wall of the vessel thereby minimizing movement of the distal tip/end during aspiration;

FIG. 8A is a distal section of while still another example aspiration catheter in accordance with the present disclosure secured within a lumen of an outer sheath via a living hinge; wherein the aspiration catheter is depicted during delivery to the target site prior to the application of aspiration;

FIG. 8B is a distal section of the example aspiration catheter in FIG. 8A subject to aspiration depicting the aspiration catheter isolated from the outer sheath by the living hinge so axial movement (e.g., back and forth) of the aspiration catheter when subject to aspiration is not transmitted to the outer sheath;

FIG. 9A depict another example aspiration catheter in accordance with the present disclosure in which along a distal section thereof is an inflatable balloon having a knurled outer surface as a stabilizing member in accordance with the present disclosure; wherein the inflatable balloon is illustrated in a non-stabilizing (i.e., non-inflated) state while being delivered through the vessel to the target site prior to application of aspiration;

FIG. 9B depicts the example aspiration catheter of FIG. 9B, wherein the external inflatable balloon having the knurled outer surface is illustrated in a stabilizing (i.e., inflated) state positioned at the target site during aspiration;

FIG. 9C is a radial cross-sectional view along line IX(C)-IX(C) through the external inflatable balloon having the knurled surface in FIG. 9B, wherein the external inflatable balloon is illustrated in the stabilizing (i.e., inflated) state;

FIG. 9D is a modification of the example in FIG. 9A, rather than an external inflatable balloon a distal section of the outer surface of the aspiration catheter itself has a knurled surface;

FIG. 10A depicts a distal section of still yet another example of an aspiration catheter in accordance with the present disclosure nested within an outer guide secured together by an elastomeric polymer forming an air gap therebetween acting as the stabilizing structural member permitting but limiting movement of the distal tip/end during aspiration;

FIG. 10B is a radial cross-sectional view along lines X(B)-X(B) of the aspiration catheter of FIG. 10A; and

FIG. 11 is an example aspiration catheter system including a vacuum pump connected in fluid communication to an aspiration catheter delivered to a target site prior to capturing/ingesting a clot therein, wherein the aspiration catheter may include any of the stabilizing structural members illustrated above.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

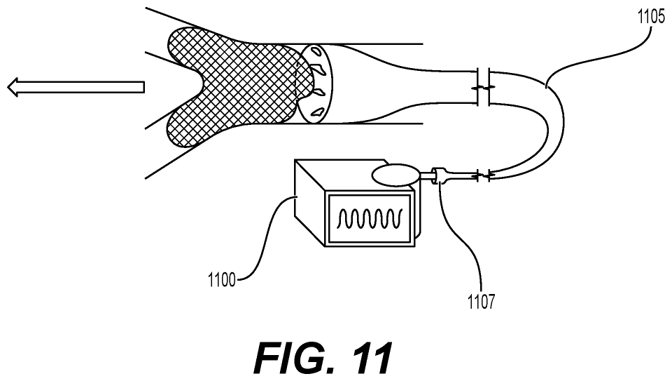

FIG. 11 illustrates the aspiration catheter 1105 having a proximal hub 1107 in fluid communication with an aspiration (e.g., vacuum or pulsatile/cyclic) pump 1100 while being delivered through the vessel to the target site of a clot prior to applying aspiration (e.g., vacuum or pulsatile/cyclic). Application of aspiration (e.g., vacuum or pulsatile/cyclic) causes excessive movement of the distal tip/end of the aspiration catheter 1105 during capture and removal of a clot posing a significant risk of damage to the inner wall of the vessel. The present disclosure is directed to an improved aspiration catheter configured to minimize the potential risk of damage to the inner wall of the vessel when subject to aspiration (e.g., vacuum (i.e., non-cyclic) or pulsatile/cyclic). One way described herein to minimize the potential risk of damage to the inner wall of the vessel in accordance with the present disclosure is to stabilize the aspiration catheter relative to the inner wall of the vessel in order to minimize excessive axial movement of the distal tip/end during aspiration. It is also recognized that there is a risk of potential damage to the inner wall of the vessel resulting from overexpansion of a radially enlargeable distal section (e.g., funnel distal section) of the aspiration catheter when ingesting a clot exceeding in diameter that of the funnel distal section and possibly even becoming stuck in the vessel. This potential risk of damage to the inner wall of the vessel is addressed in accordance with the present disclosure by limiting or restricting overexpansion (i.e., radial widening) of a radially enlargeable distal section of the aspiration catheter.

Several non-limiting examples of stabilizing structural members for stabilizing the aspiration catheter relative to the inner wall of the vessel to minimize movement of the distal tip/end of the aspiration catheter when subject to aspiration (e.g., vacuum or pulsatile/cyclic) are illustrated and described below.

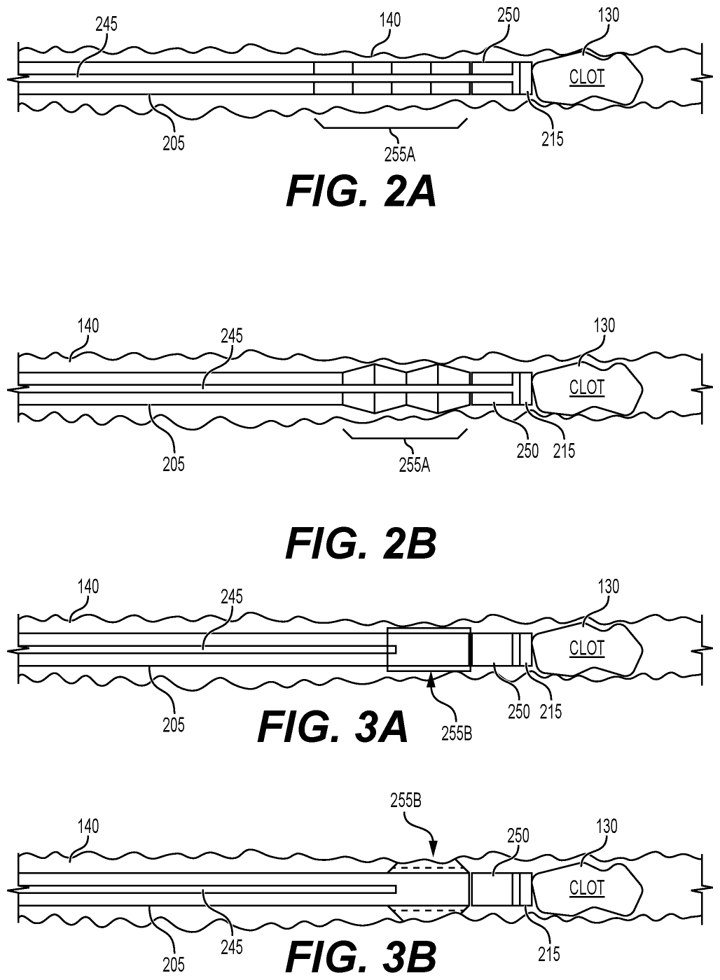

FIGS. 2A & 2B depict an example aspiration catheter in accordance with the present disclosure including a stabilizing structural member as a portion along the distal section of the aspiration catheter proximally of the distal tip/end that expands radially when actuated or deployed via a pulling member (e.g., pull wire). The aspiration catheter 205 in FIG. 2A is depicted while being delivered through the vessel to the target site on the proximal side of the target clot prior to applying aspiration (e.g., vacuum or pulsatile/cyclic). Pulling member 245 (e.g., a wire) extends axially through the lumen of the aspiration catheter 205 while being secured (e.g., collar or anchor) at its distal end within the distal section of the aspiration catheter 205. In the example in FIGS. 2A & 2B the stabilizing member is an axially compressible section 255a (e.g., a bellows section).

In operation, when being delivered through the vessel to the target site on a proximal side of the clot the pulling member 245 (e.g., wire) is not actuated (i.e., not pulled in a proximal direction) so that the axially compressible section 255a (e.g., bellows section) remains in a default non-stabilizing (i.e., radially contracted) state. Thus, the aspiration catheter 205 including the axially compressible section 255a has an approximately uniform outer diameter trackable to navigate through the vessel to the target site (FIG. 2A). Once the aspiration catheter is delivered to the target site in the vessel aspiration (e.g., vacuum or pulsatile/cyclic) is applied to capture the clot. The pulling member 245 (e.g., wire) is actuated by pulling in the proximal direction. In turn, the axially compressible section 255a (e.g., bellows section) transitions to a stabilizing state in which portions thereof expand radially (similar to that of an accordion) physically contacting the inner wall of the vessel thereby stabilizing the distal tip/end of the aspiration catheter when subject to aspiration (FIG. 2B).

Another example of the aspiration catheter in accordance with the present disclosure is provided in FIGS. 3A & 3B wherein the stabilizing member is an external radially expandable sleeve (e.g., an external inflatable balloon sleeve 255b) disposed externally along the distal section of the aspiration catheter 205 proximally of the distal tip/end. FIG. 3A depicts the external inflatable balloon sleeve 255b while in a non-stabilizing (i.e., deflated) state prior to receiving an inflation media via an inflation lumen 245′. While in this non-stabilizing (i.e., deflated) state illustrated in FIG. 3A, the aspiration catheter 205 may be delivered through the vessel to the target site on the proximal side of the clot. Once properly positioned in the vessel on the proximal side of the clot aspiration (e.g., vacuum or pulsatile/cyclic) is applied to capture the clot. Prior to, simultaneously with, or following initiating aspiration, inflation media is delivered via the inflation lumen 245′ transitioning the external inflatable balloon sleeve 255b to a stabilizing (i.e., inflated) state directly physically contacting the inner wall of the vessel stabilizing the aspiration catheter relative to the inner wall of the vessel thereby minimizing movement of the distal tip/end of the aspiration catheter when subject to aspiration (FIG. 3B).

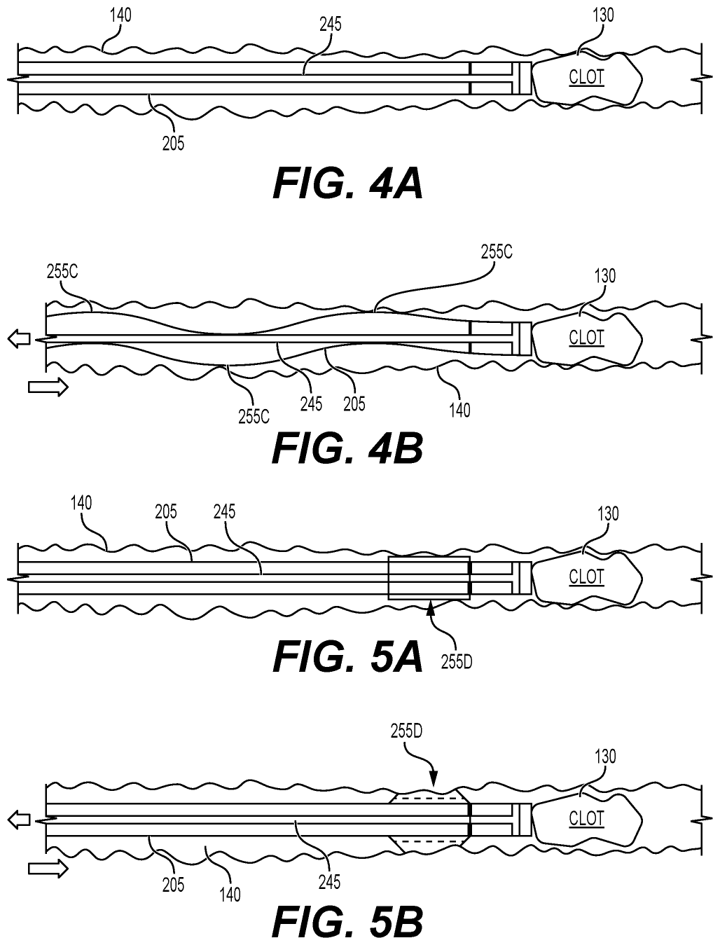

In yet another example of the aspiration catheter in accordance with the present disclosure the stabilizing member is provided by the intentional altering of its overall longitudinal/axial shape in response to actuating (i.e., pulling in an axial direction) a pulling member. The term “altering overall longitudinal/axial shape” when referred to in the present disclosure is defined as the aspiration catheter having a central longitudinal axis that no longer is equidistant from the inner wall of the lumen. In the example depicted in FIG. 4B, the altered overall longitudinal/axial shape depicted is snaking, spiraling, or buckling on alternating sides of the aspiration catheter. Referring to FIG. 4A the aspiration catheter 205 has a pulling member 245 (e.g., wire) secured at its distal end to the distal section of the aspiration catheter and extending in a proximal direction through the lumen. The aspiration catheter 205 in FIG. 4A is depicted in a non-stabilizing state (i.e., unaltered overall axial/longitudinal shape in which the central longitudinal axis extending therethrough is substantially equidistant from the inner wall of the lumen). In response to the actuating (i.e., pulling in a proximal direction) of the pulling member (e.g., wire), the aspiration catheter 205 transitions to a stabilizing state (i.e., altered overall axial/longitudinal shape in which the central longitudinal axis extending therethrough is no longer equidistant from the inner wall of the lumen).

In operation, while being delivered through the vessel to the target site on a proximal side of the clot the pulling member 245 (e.g., wire) is not actuated (i.e., not pulled in a proximal direction) so that the aspiration catheter 205 remains in a default non-stabilizing (i.e., unaltered overall axial/longitudinal shape in which the central longitudinal axis extending therethrough is substantially equidistant from the inner wall of the lumen). Once the aspiration catheter 205 is delivered to the target site in the vessel, aspiration (e.g., vacuum or pulsatile/cyclic) is applied to capture the clot. In response to actuating the pulling member the overall axial/longitudinal shape of the aspiration catheter 205 itself is altered (e.g., snakes, spirals, or buckles) so that the central longitudinal axis therethrough is no longer equidistant from the inner wall of the lumen. Those regions in which the aspiration catheter buckles, bends, or curves 255c directly physically contact the inner wall of the vessel thereby stabilizing the aspiration catheter in response to excessive movement of the distal tip/end of the aspiration catheter when subject to aspiration (FIG. 4B).

While FIGS. 5A & 5B illustrate still another example of the aspiration catheter in accordance with the present disclosure in which the stabilizing member is a prefilled external sleeve disposed along the distal section of the aspiration catheter proximally of the distal tip/end. In response to exerting an axial compressive force the prefilled external sleeve expands radially. Referring to FIG. 5A, an external sleeve 255d is disposed about the distal section of the aspiration catheter 205 proximally of its distal tip/end. The external sleeve 255d is prefilled with a deformable material (e.g., a biocompatible gel material). A pulling member 245 (e.g., a wire) extends axially through the lumen of the aspiration catheter 205 while being secured at its distal end within the distal section of the aspiration catheter 205. In response to actuation (e.g., pulling in the proximal direction) of the pulling member 245 (e.g., wire), the prefilled external sleeve along with the deformable material contained therein is longitudinally/axially compressed expanding radially.

In operation, when being delivered through the vessel to the target site on a proximal side of the clot the pulling member 245 (e.g., wire) is not actuated (i.e., not pulled in a proximal direction) so that the prefilled external sleeve 255d remains in a default non-stabilizing state (i.e., minimum outer diameter). Thus, the aspiration catheter 205 along the prefilled external sleeve 255d secured thereto is trackable to navigate through the vessel to the target site (FIG. 5A). Once the aspiration catheter is delivered to the target site in the vessel, aspiration (e.g., vacuum or pulsatile/cyclic) is applied to capture the clot. Simultaneously therewith or prior to applying the aspiration the pulling member 245 (e.g., wire) is pulled in the proximal direction. In turn, the prefilled external sleeve 225d and deformable material contained therein when axially compressed expands radially transitioning to a stabilizing state (i.e., maximum outer diameter) physically contacting the inner wall of the vessel thereby stabilizing the aspiration catheter relative to the inner wall of the vessel while minimizing movement of the distal tip/end when subject to aspiration (FIG. 5B).

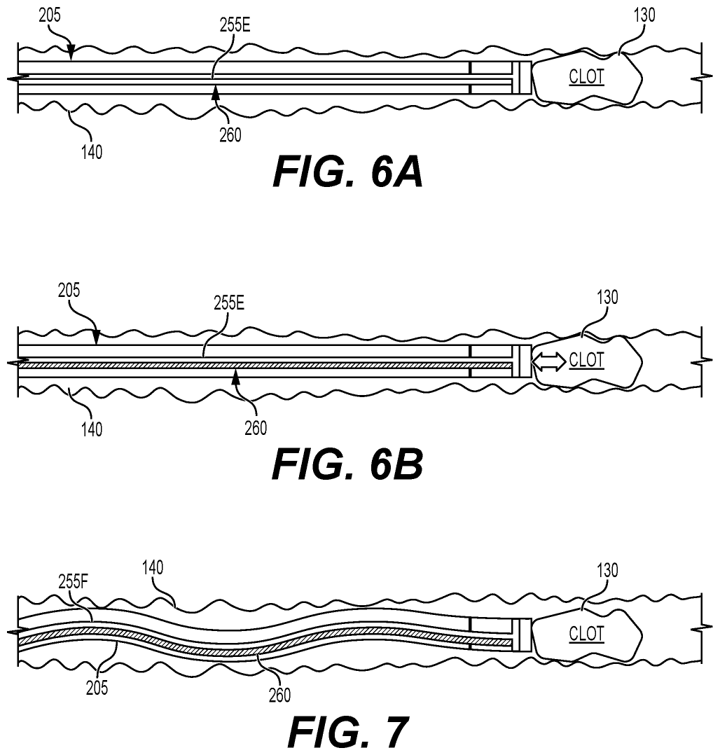

FIGS. 6A & 6B depict non-stabilizing and stabilizing states, respectively, of yet another example of the stabilizing structural member of an aspiration catheter in accordance with the present disclosure. As an alternative to actuating the stabilizing structural member by axially pulling on a pulling member imposing an axially compressive force on the aspiration catheter, in this example the stabilizing member is thermally actuated. Specifically, the aspiration catheter 205 has associated therewith a nitinol wire 255e (i.e., nickel titanium alloy) that when thermally activated by an electrical circuit 260 to a specified temperature range alters the characteristics or properties of the nitinol wire 255e. Referring to the example in FIG. 6A, the nitinol wire 255e and electrical circuit 260 (e.g., conductive metal wire) are both disposed within the lumen of the aspiration catheter 205.

In operation, while being delivered through the vessel to the target site on a proximal side of the clot, the nitinol wire 255e is in a non-stabilizing state (i.e., a Martensitic phase—temperature below Austenite transformation finish temperature (A_{f})) so that the nitinol wire is compliant and readily trackable able to navigate through the vessel to the target site. Once the aspiration catheter is delivered to the target site in the vessel, aspiration (e.g., vacuum or pulsatile/cyclic) is applied to capture the clot. In advance of or simultaneously while applying aspiration, in response to thermal actuation (i.e., thermal heating) the nitinol wire 255e transitions to a stabilizing state (i.e., Austenite phase—temperature heated above the Austenite transformation finish temperature (A_{f})) thereby stiffening the nitinol wire 255e limiting, reducing, or curtailing excessive movement of the distal tip/end of the aspiration catheter resulting from aspiration (e.g., vacuum or pulsatile/cyclic, preferably pulsatile/cyclic). As a modification, when thermally actuated (i.e., thermally heated) the nitinol wire in a stabilizing state (i.e., Austenite phase—temperature heated above the Austenite transformation finish temperature (A_{f})) may be set or formed to have a curved, undulating, or spiral shape in a longitudinal/axial direction, as depicted in FIG. 7. As a result, stability provided by the stiffening of the form spiral or waveform shape nitinol wire 255e while in the Austenitic phase is fostered, supplemented, combined, or enhanced, by the bent regions of the aspiration catheter in direct physical contact with the inner wall of the lumen.

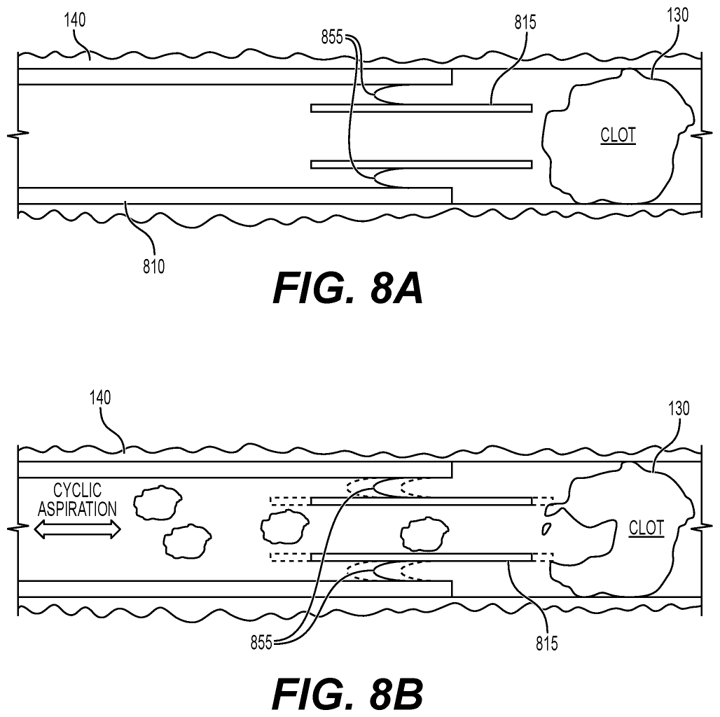

A still different approach to imparting stability to the aspiration catheter while subject to aspiration (e.g., vacuum or pulsatile/cyclic) in accordance with the present disclosure is illustrated in FIGS. 8A and 8B employing a flexible living hinge. An aspiration catheter 815 is disposed within a lumen of an outer sheath 810 arranged proximally thereto and connected via a living hinge 855. Living hinge 855, preferably made of a flexible biocompatible polymer, isolates axial movement of the aspiration catheter 815 from that of the outer sheath 810 while still allowing axial movement of the aspiration catheter 815 when subject to aspiration (e.g., vacuum or pulsatile/cyclic). The outer sheath 810 in which the aspiration catheter 815 is disposed provides greater stability to curtail excessive axial movement of the distal section 815 of the aspiration catheter relative to that provided by the inner wall of the vessel. The application of aspiration (e.g., vacuum or pulsatile/cyclic, but preferably pulsatile/cyclic aspiration as denoted by the bidirectional arrows) imparts axial movement to the aspiration catheter 815 accommodated by the living hinge 855 (as denoted by the dashed lines). Excessive axial movement of the distal section 815 is curtailed (i.e. . . . , without preventing or prohibiting all axial movement) by the living hinge 855, hereinafter referred to as curtailed (i.e., non-excessive) axial movement. Vibration together with curtailed axial movement of the distal section 815 relative to the outer sheath 810 assists in tearing up the clot (as depicted by the smaller torn off pieces of clot) improving the efficiency of capture and removal the clot (especially fibrin rich and/or oversized clots).

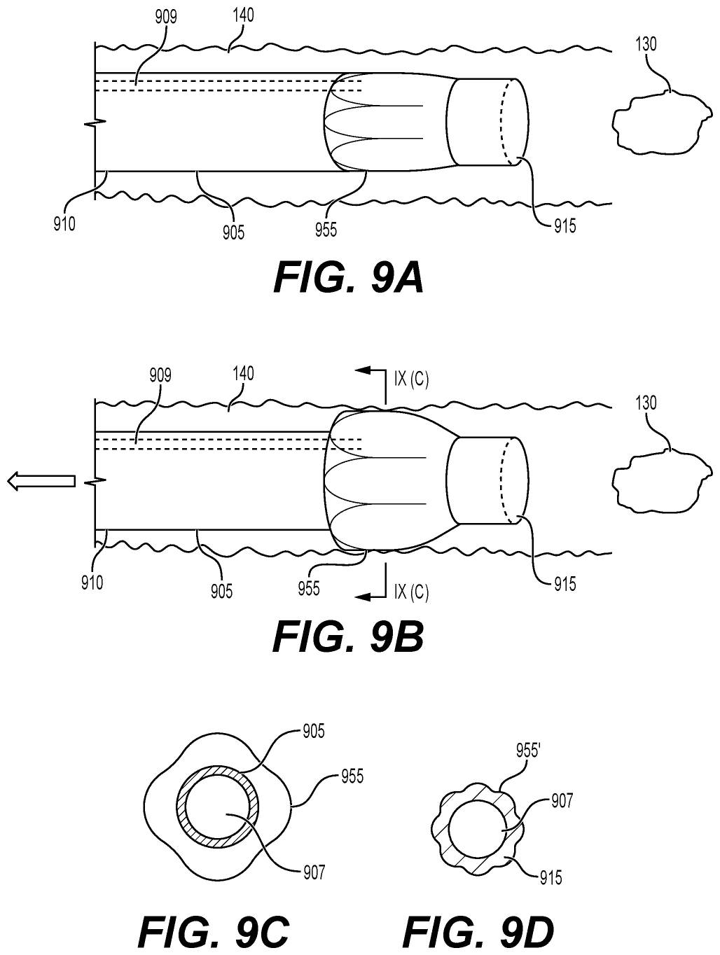

The stabilizing structural member may also be a non-circular (e.g., knurled) surface, either a separate component attached thereto or a section of the aspiration catheter itself, that is disposed along a distal section proximally of the distal tip/end. FIGS. 9A-9C illustrate an example of an inflatable sleeve 955 disposed along a distal section of the aspiration catheter 905 proximally of the distal tip/end 915, the inflatable sleeve 955 having a knurled outer surface when inflated (FIG. 9C). Sleeve 955 is inflatable via inflation media received through an inflation lumen 909. Prior to being inflated, sleeve 955 is in a non-stabilizing (i.e., minimum outer diameter) state so that the aspiration catheter 905 may be tracked and navigated through the vessel to the target site. Once positioned with the distal tip/end 915 of the aspiration catheter 905 on the proximal side of the target clot 130, aspiration (e.g., vacuum or pulsatile/cyclic) is applied to capture the clot therein. Either before or simultaneously with applying aspiration, sleeve 955 is inflated by filling with inflation media via the inflation lumen 909. In an inflated (i.e., stabilizing) state, the outer surface of the sleeve 955 has a knurled contour (FIGS. 9B & 9C) stabilizing the aspiration catheter in response to excessive movement of the distal tip/end during aspiration. By way of example only, the outer surface of the sleeve 955 in FIGS. 9B & 9C has four ridges or bumps, but may be modified, as desired, to have any number of two or more bumps. The greater the number of bumps the larger the contact surface area with the inner wall of the vessel and hence increased stability of the distal tip/end of the aspiration catheter. As mentioned above, as an alternative to having an inflatable sleeve having a knurled outer surface, a portion of the aspiration catheter itself along its distal section proximally of the distal tip/end may have a knurled outer contour or surface, as depicted in FIG. 9D. Once again, the number of ridges or bumps may be modified, as desired.

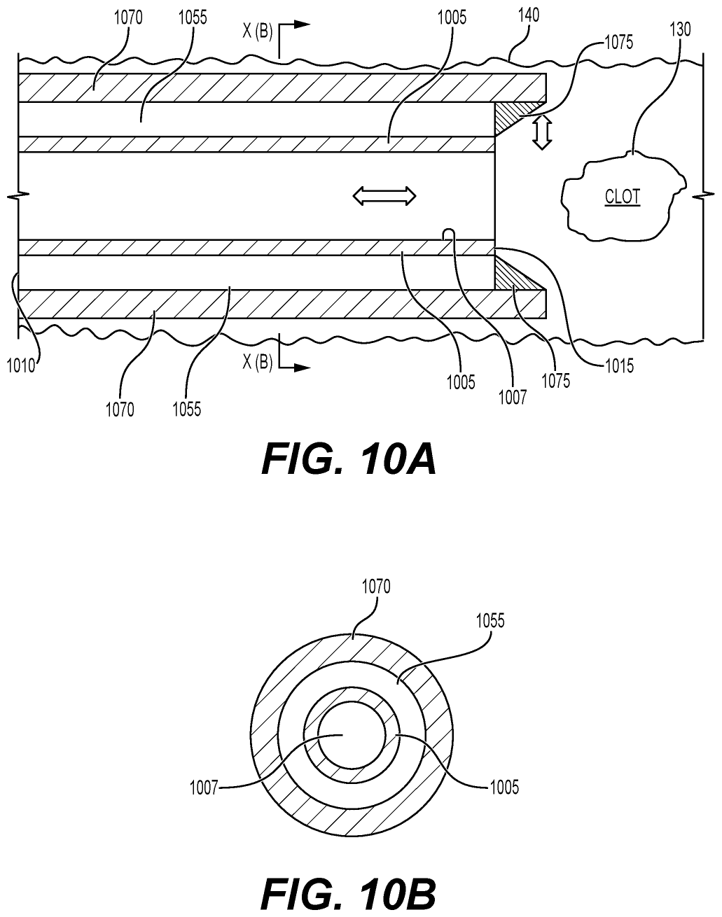

In FIGS. 10A & 10B the aspiration catheter 1005 is stabilized in response to excessive movement of the distal tip/end 1015 by insulating the aspiration catheter 1005 within an outer sheath 1070 with a stabilizing member (i.e., absorbing layer) disposed therebetween. In the example of FIGS. 10A & 10B, the absorbing layer is an air gap 1055 disposed between the outer sheath 1070 and the aspiration catheter 1005 secured to one another via a soft elastomeric polymer 1075. The air gap 1055 stabilizes the aspiration catheter 1005 in response to excessive movement of the distal tip/end 1015 during aspiration while the soft elastomeric polymer 1075 imparts an elastomeric returning force restoring the aspiration catheter 1005 to a default position within the outer sleeve 1070. In the preferred example depicted in FIGS. 10A & 10B, the default position of the aspiration catheter is coaxially centered within the outer sleeve 1070.

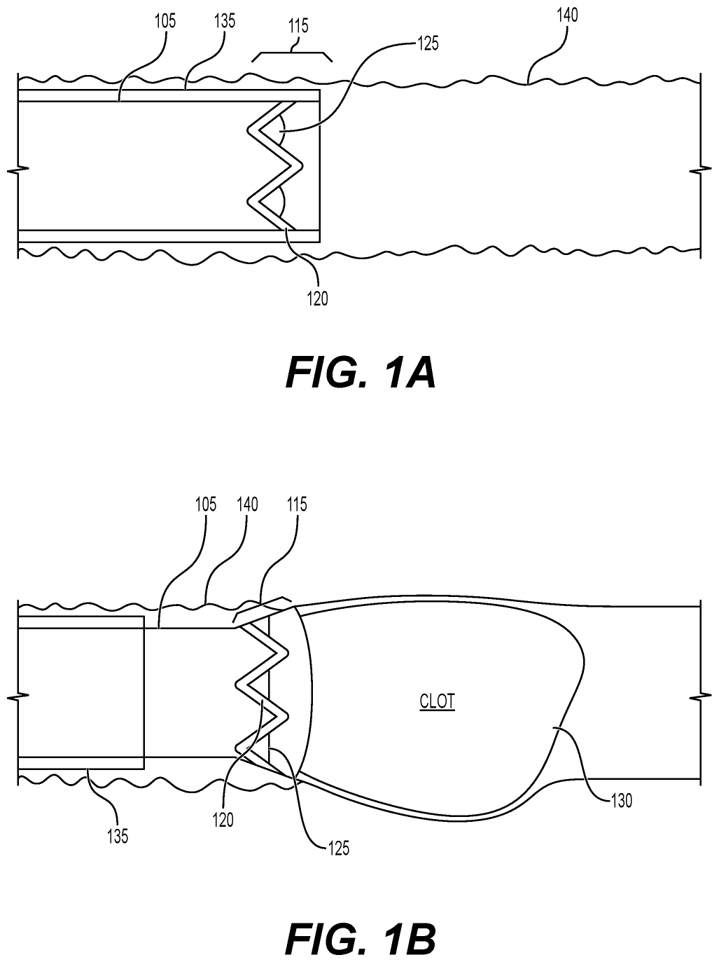

In the preceding examples, the risk of possible damage to the inner wall of the vessel resulting from excessive axial movement of the distal tip/end of the aspiration catheter during aspiration is minimized by stabilizing the aspiration catheter relative to the inner wall of the vessel. There is also a risk of damage to the inner wall of the vessel during radial expansion of the distal tip/end of the aspiration catheter during capture of a targeted clot. To assist in capture of the clot, the aspiration catheter 105 may include a radially self-expandable distal section 115 (e.g., distal funnel section) transitionable from a non-deployed state (i.e., radially compressed/contracted) while sheathed within an outer guide or sheath 135 to a deployed state (i.e., radially expanded/enlarged) when unsheathed from the outer guide or sheath 135. Risk of potential damage to the inner wall of the vessel 140 and possibly eventually becoming stuck therein may occur due to undesirable radial overexpansion of the radially self-expandable distal section 115 (e.g., enlargeable distal funnel section). Such undesirable radial overexpansion poses a significant risk when ingesting into the aspiration catheter an oversized clot (wherein the outer profile of the clot exceeds that of the inner diameter of the radially self-expandable distal section while in the deployed state)(as shown in FIG. 1B). FIGS. 1A & 1B depict an exemplary aspiration catheter 105 with a radially self-expandable distal section 115. Preferably, a frame or skeleton 120, e.g., made of nitinol, is provided along at least a portion, preferably along the entire, distal edge of the self-expandable distal section 115 of the aspiration catheter 105 to maintain a maximum outer diameter while ingesting the clot therein. FIG. 1A depicts the aspiration catheter 105 with the radially self-expandable distal section 115 in a non-deployed state while sheathed within the outer guide or sheath 135. It is while in the non-deployed state shown in FIG. 1A that the aspiration catheter 105 is delivered through the vessel 140 to the target site on the proximal side of the clot 130. Delivery of the outer guide 135 and aspiration catheter 105 to the target site in the vasculature may occur either in series (i.e., the outer guide 135 navigated first followed by advancement in a distal direction of the aspiration catheter 105 therein) or simultaneously together at the same time. Once properly positioned at the target site, the aspiration catheter 105 is advanced (i.e., pushed) in a distal direction until the distal section 115 emerges from the distal end of the outer guide 135. When unsheathed, the radially self-expandable distal section 115 automatically transitions from the non-deployed state to the deployed state (FIG. 1B). In accordance with the present disclosure, the tether 125 (e.g., nitinol wire) acts as a stabilizing structural member to limit, restrict, or curtail the extent of radial expansion (i.e., prevent overexpansion) of the radially self-expandable distal section 115 when ingesting the clot 130 therein thereby minimizing the risk of damage to the inner wall of the vessel 140.

Aspects of the disclosure are also provided by the following numbered clauses:

### Clause 1

An aspiration catheter comprising: a body (105, 205, 815, 905, 1005) having a proximal end and an opposite distal tip with a lumen extending in a longitudinal direction therethrough from the proximal end to the distal tip; and a stabilizing structural member (125, 255a, 255b, 255c, 255d, 255e, 255f, 855, 955, 955′, 1055) stabilizing the body (105, 205, 855, 905, 1005) to minimize movement and/or radial expansion of the distal tip of the body (105, 205, 815, 905, 1005) while subject to aspiration.

### Clause 2

The aspiration catheter of Clause 1, further comprising: an actuating member (245, 260) altering a parameter associated with the body (205) when transitioning from a non-actuated state to an actuated state; wherein, when the actuating member (245, 260) transitions from the non-actuated state to the actuated state, the stabilizing structural member (255a, 255b, 255c, 255d, 255e, 255f) transitions from a non-stabilizing state to a stabilizing state stabilizing the body (205) to minimize movement of the distal tip of the body (205) when subject to aspiration.

### Clause 3

The aspiration catheter of Clause 2, wherein the actuating member is a pulling member (245) extending longitudinally in the lumen fixedly secured to a distal section proximally of the distal tip; and the pulling member (245) is pullable in a proximal direction altering the parameter of longitudinal length of the body (205) transitionable from a longitudinally elongated state when the pulling member (245) is in the non-actuated state and longitudinally contracted state when the pulling member (245) is in the actuated state; and the stabilizing structural member (255a, 255b, 255c, 255d) expanding radially when the pulling member (245) is in the actuated state with the body (205) in the longitudinally contracted state stabilizing the body (205) minimizing movement of the distal tip when subject to aspiration.

### Clause 4

The aspiration catheter of Clause 3, wherein the stabilizing structural member is: (i) buckling (255c) of the body (205) on alternating sides of a longitudinal centerline when the pulling member (245) is in the actuated state with the body (205) in the longitudinally contracted state; (ii) a longitudinally compressible bellows section (255a) of the body (205) with a plurality of discrete portions therein expandable radially when the pulling member (245) is in the actuated state with the body (205) in the longitudinally contracted state; or (iii) a sleeve (255b, 255d), inflatable or prefilled with deformable matter, disposed externally about the body (205) proximally of the distal tip.

### Clause 5

The aspiration catheter of Clause 2, wherein the stabilizing structural member is a nitinol wire (255e, 255f) associated with the body (205) and the actuating member is an electrical circuit (260) altering the parameter of temperature transitionable from a non-actuated state at a temperature below Austenite transformation finish temperature to an actuated state at a temperature above Austenite transformation finish temperature; wherein when the electrical circuit (260) is in the non-actuated state at the temperature below the Austenite transformation finish temperature the nitinol wire (255e, 255f) being in a Martensite phase having a lower stiffness state, whereas when the electrical circuit (260) is in the actuated state at the temperature above the Austenite transformation finish temperature the nitinol wire (255e, 255f) being in a Austenite phase having a higher stiffness stabilizing the body (205) minimizing movement of the distal tip of the body (205) when subject to the aspiration.

### Clause 6

The aspiration catheter of Clause 5, wherein while in the Austenite phase, the nitinol wire is shape set to a curved, undulating, or spiral shape.

### Clause 7

The aspiration catheter of Clause 1, wherein the body (815) is disposed within an outer sheath (810) connected thereto via a living hinge (855) acting as the stabilizing structural member isolating the distal tip of the body (815) from the outer sheath (810) so that the axial movement of the body (815) while subject to the aspiration is not transmittable to the outer sheath (810).

### Clause 8

The aspiration catheter of Clause 1, wherein the stabilizing structural member is a knurled outer surface (955′) of a portion of the body (905) itself or an inflatable balloon (955) disposed thereabout.

### Clause 9

The aspiration catheter of Clause 1, further comprising an outer sleeve (1070) disposed about the body (1005) and secured thereto via an elastomeric polymer defining an air gap therebetween acting as the stabilizing structural member stabilizing the body (1005) minimizing movement of the distal tip of the body (1005) when subject to aspiration; wherein the elastomeric polymer restores the body (1005) to a position substantially centered within the outer sleeve (1070).

### Clause 10

The aspiration catheter of any of Clauses 1 through 9, wherein the body (105) of the aspiration catheter has a radially self-expandable distal section (115) including the distal tip; wherein the stabilizing structural member is a tether (125) associated with the radially self-expandable distal section (115) restricting the radial expansion thereof while subject to the aspiration to prevent overexpansion.

### Clause 11

A method for using an aspiration catheter including a body (105, 205, 815, 905, 1005) having a proximal end and an opposite distal tip with a lumen extending in a longitudinal direction therethrough from the proximal end to the distal tip; and a stabilizing structural member (125, 255a, 255b, 255c, 255d, 255e, 255f, 855, 955, 955′, 1055) stabilizing the body (105, 205, 855, 905, 1005) to minimize movement and/or radial expansion of the distal tip of the body (105, 205, 815, 905, 1005) while subject to aspiration; the method comprising the steps of: navigating the aspiration catheter through a vessel to a target site with the distal tip positioned on a proximal side of a targeted clot; applying aspiration to the body (105, 205, 815, 905, 1005) to capture the clot into the lumen; and simultaneously while applying the aspiration, stabilizing the body (105, 205, 815, 905, 1005) relative to an inner wall of the vessel via the stabilizing structural member (125, 255a, 255b, 255c, 255d, 255e, 255f, 855, 955, 955′, 1055) in order to minimize the movement and/or the radial expansion of the distal tip during the aspiration to minimize risk of damage to the inner wall of the vessel.

### Clause 12

The method of Clause 11, wherein the stabilizing step comprises altering a parameter associated with the body (205) in response to an actuating member (245, 260) associated with the body (205) transitioning from a non-actuated state to an actuated state; and when the actuating member (245, 260) transitions from the non-actuated state to the actuated state, the stabilizing structural member (255a, 255b, 255c, 255d, 255e, 255f) transitions from a non-stabilizing state to a stabilizing state stabilizing the body (205) to minimize movement of the distal tip of the body (205) when subject to aspiration.

### Clause 13

The method of Clause 12, wherein the altered parameter is longitudinal length of the body; and the actuating member is a pulling member (245) extending longitudinally in the lumen fixedly secured to the distal section; wherein the stabilizing of the axial movement step comprises: altering the longitudinal length of the body (205) in response to pulling in a proximal direction the pulling member (245), the body (205) transitioning from a longitudinally elongated state when the pulling member (245) is in the non-actuated state to a longitudinally contracted state when the pulling member (245) is in the actuated state; and in response to the altering of the longitudinal length of the body (205) when the pulling member (245) is in the actuated state with the body (205) in the longitudinally contracted state, expanding radially the stabilizing structural member (255a, 255b, 255c, 255d) stabilizing the body (205) minimizing movement of the distal tip when subject to aspiration.

### Clause 14

The method of Clause 13, wherein the stabilizing structural member is: (i) buckling of the body (205) on alternating sides of a longitudinal centerline when the pulling member (245) is in the actuated state with the body (205) in the longitudinally contracted state; (ii) a longitudinally compressible bellows section (255a) of the body (205) with a plurality of discrete portions expandable radially when the pulling member (245) is in the actuated state with the body (205) in the longitudinally contracted state; or (iii) a sleeve (255b, 255d), inflatable or prefilled with deformable matter, disposed externally about the body (205) proximally of the distal tip.

### Clause 15

The method of Clause 12, wherein the stabilizing structural member is a nitinol wire (255e, 255f) associated with the body (205) and the actuating member is an electrical circuit (260) altering the parameter of temperature transitionable from a non-actuated state at a temperature below Austenite transformation finish temperature to an actuated state at a temperature above Austenite transformation finish temperature; wherein the step of stabilizing the body (205) relative to the inner wall of the vessel comprises: increasing the temperature of the nitinol wire (255e, 255f) via the electrical circuit (260) transitioning from the non-actuated state at the temperature below the Austenite transformation finish temperature to the actuated state at the temperature above the Austenite transformation finish temperature; and in response to increasing the temperature of the nitinol wire (255e, 255f) via the electrical circuit (260), increasing stiffness of the nitinol wire (255e, 255f) in the body (205) from a lower stiffness state being in a Martensite phase when the temperature of the body (205) is below the Austenite transformation finish temperature to a higher stiffness state being in a Austenite phase when the temperature of the nitinol wire (255e, 255f) is above the Austenite transformation finish temperature; wherein the higher stiffness state of the nitinol wire (255e, 255f) in the body (205) acting as the stabilizing structural member stabilizing the body (205) in order to minimize movement of the distal tip when applying the aspiration.

### Clause 16

The method of Clause 15, wherein while in the Austenite phase, the nitinol wire is shape set to a curved, undulating, or spiral shape.

### Clause 17

The method of Clause 11, wherein the body (815) is disposed within an outer sheath (810) connected thereto via a living hinge (855) acting as the stabilizing structural member isolating the distal tip of the body (815) from the outer sheath (810) so that the axial movement of the body (815) while applying the aspiration is not transmittable to the outer sheath (810).

### Clause 18

The method of Clause 11, wherein the stabilizing structural member is a knurled outer surface (955′) of a portion of the body (905) itself or an inflatable balloon (955) disposed thereabout.

### Clause 19

The method of Clause 11, wherein the aspiration catheter further comprises an outer sleeve (1070) disposed about the body (1005) and secured together via an elastomeric polymer (1075) defining an air gap (1055) therebetween serving as the stabilizing structural member stabilizing the body (1005) minimizing movement of the distal tip when applying the aspiration; wherein the elastomeric polymer (1075) restores the body (1005) to a position substantially centered within the outer sleeve (1070).

### Clause 20

The method of any of Clauses 11 through 19, wherein the navigating step comprises the steps of: advancing the aspiration catheter through an outer guide (135) in the distal direction towards the targeted clot; and engaging the targeted clot with the aspiration catheter while subject to aspiration and radially self-transitioning the radially enlargeable distal section from a radially contracted state to a radially expanded state; wherein the stabilizing step comprises preventing the radial overexpansion of the distal tip of the body via a tether (125) acting as the stabilizing structural member to minimize risk of damage to the inner wall of the vessel.

The descriptions contained herein are examples and are not intended in any way to limit the scope of the present disclosure. As described herein, the present disclosure contemplates many variations and modifications of the aspiration catheter that reduces the risk of damage to the inner wall of the vessel during aspiration. Modifications and variations apparent to those having skilled in the pertinent art according to the teachings of this disclosure are intended to be within the scope of the claims which follow.

What is claimed is:

1. An aspiration catheter comprising:
a body having a proximal end and an opposite distal tip with a lumen extending in a longitudinal direction therethrough from the proximal end to the distal tip; and
a stabilizing structural member stabilizing the body to minimize movement and/or radial expansion of the distal tip of the body while subject to aspiration.

2. The aspiration catheter in accordance with claim 1, further comprising:
an actuating member altering a parameter associated with the body when transitioning from a non-actuated state to an actuated state;
wherein, when the actuating member transitions from the non-actuated state to the actuated state, the stabilizing structural member transitions from a non-stabilizing state to a stabilizing state stabilizing the body to minimize movement of the distal tip of the body when subject to aspiration.

3. The aspiration catheter in accordance with claim 2, wherein the actuating member is a pulling member extending longitudinally in the lumen fixedly secured to a distal section proximally of the distal tip; and the pulling member is pullable in a proximal direction altering the parameter of longitudinal length of the body transitionable from a longitudinally elongated state when the pulling member is in the non-actuated state and longitudinally contracted state when the pulling member is in the actuated state; and the stabilizing structural member expanding radially when the pulling member is in the actuated state with the body in the longitudinally contracted state stabilizing the body minimizing movement of the distal tip when subject to aspiration.

4. The aspiration catheter in accordance with claim 3, wherein the stabilizing structural member is: (i) buckling of the body on alternating sides of a longitudinal centerline when the pulling member is in the actuated state with the body in the longitudinally contracted state; (ii) a longitudinally compressible bellows section of the body with a plurality of discrete portions therein expandable radially when the pulling member is in the actuated state with the body in the longitudinally contracted state; or (iii) a sleeve, inflatable or prefilled with deformable matter, disposed externally about the body proximally of the distal tip.

5. The aspiration catheter in accordance with claim 2, wherein the stabilizing structural member is a nitinol wire associated with the body and the actuating member is an electrical circuit altering the parameter of temperature transitionable from a non-actuated state at a temperature below Austenite transformation finish temperature to an actuated state at a temperature above Austenite transformation finish temperature; wherein when the electrical circuit is in the non-actuated state at the temperature below the Austenite transformation finish temperature the nitinol wire being in a Martensite phase having a lower stiffness state, whereas when the electrical circuit is in the actuated state at the temperature above the Austenite transformation finish temperature the nitinol wire being in a Austenite phase having a higher stiffness stabilizing the body minimizing movement of the distal tip of the body when subject to the aspiration.

6. The aspiration catheter in accordance with claim 5, wherein while in the Austenite phase, the nitinol wire is shape set to a curved, undulating, or spiral shape.

7. The aspiration catheter in accordance with claim 1, wherein the body is disposed within an outer sheath connected thereto via a living hinge acting as the stabilizing structural member isolating the distal tip of the body from the outer sheath so that the axial movement of the body while subject to the aspiration is not transmittable to the outer sheath.

8. The aspiration catheter in accordance with claim 1, wherein the stabilizing structural member is a knurled outer surface of a portion of the body itself or an inflatable balloon disposed thereabout.

9. The aspiration catheter in accordance with claim 1, further comprising an outer sleeve disposed about the body and secured thereto via an elastomeric polymer defining an air gap therebetween acting as the stabilizing structural member stabilizing the body minimizing movement of the distal tip of the body when subject to aspiration; wherein the elastomeric polymer restores the body to a position substantially centered within the outer sleeve.

10. The aspiration catheter in accordance with claim 1, wherein the body of the aspiration catheter has a radially self-expandable distal section including the distal tip; wherein the stabilizing structural member is a tether associated with the radially self-expandable distal section restricting the radial expansion thereof while subject to the aspiration to prevent overexpansion.

11. A method for using an aspiration catheter including a body having a proximal end and an opposite distal tip with a lumen extending in a longitudinal direction therethrough from the proximal end to the distal tip; and a stabilizing structural member stabilizing the body to minimize movement and/or radial expansion of the distal tip of the body while subject to aspiration; the method comprising the steps of:
navigating the aspiration catheter through a vessel to a target site with the distal tip positioned on a proximal side of a targeted clot;
applying aspiration to the body to capture the clot into the lumen; and
simultaneously while applying the aspiration, stabilizing the body relative to an inner wall of the vessel via the stabilizing structural member in order to minimize the movement and/or the radial expansion of the distal tip during the aspiration to minimize risk of damage to the inner wall of the vessel.

12. The method in accordance with claim 11, wherein the stabilizing step comprises altering a parameter associated with the body in response to an actuating member associated with the body transitioning from a non-actuated state to an actuated state; and when the actuating member transitions from the non-actuated state to the actuated state, the stabilizing structural member transitions from a non-stabilizing state to a stabilizing state stabilizing the body to minimize movement of the distal tip of the body when subject to aspiration.

13. The method in accordance with claim 12, wherein the altered parameter is longitudinal length of the body; and the actuating member is a pulling member extending longitudinally in the lumen fixedly secured to the distal section; wherein the stabilizing of the axial movement step comprises:
altering the longitudinal length of the body in response to pulling in a proximal direction the pulling member, the body transitioning from a longitudinally elongated state when the pulling member is in the non-actuated state to a longitudinally contracted state when the pulling member is in the actuated state; and
in response to the altering of the longitudinal length of the body when the pulling member is in the actuated state with the body in the longitudinally contracted state, expanding radially the stabilizing structural member stabilizing the body minimizing movement of the distal tip when subject to aspiration.

14. The method in accordance with claim 13, wherein the stabilizing structural member is: (i) buckling of the body on alternating sides of a longitudinal centerline when the pulling member is in the actuated state with the body in the longitudinally contracted state; (ii) a longitudinally compressible bellows section of the body with a plurality of discrete portions expandable radially when the pulling member is in the actuated state with the body in the longitudinally contracted state; or (iii) a sleeve, inflatable or prefilled with deformable matter, disposed externally about the body proximally of the distal tip.

15. The method in accordance with claim 12, wherein the stabilizing structural member is a nitinol wire associated with the body and the actuating member is an electrical circuit altering the parameter of temperature transitionable from a non-actuated state at a temperature below Austenite transformation finish temperature to an actuated state at a temperature above Austenite transformation finish temperature; wherein the step of stabilizing the body relative to the inner wall of the vessel comprises:
increasing the temperature of the nitinol wire via the electrical circuit transitioning from the non-actuated state at the temperature below the Austenite transformation finish temperature to the actuated state at the temperature above the Austenite transformation finish temperature; and
in response to increasing the temperature of the nitinol wire via the electrical circuit, increasing stiffness of the nitinol wire in the body from a lower stiffness state being in a Martensite phase when the temperature of the body is below the Austenite transformation finish temperature to a higher stiffness state being in a Austenite phase when the temperature of the nitinol wire is above the Austenite transformation finish temperature; wherein the higher stiffness state of the nitinol wire in the body acting as the stabilizing structural member stabilizing the body in order to minimize movement of the distal tip when applying the aspiration.

16. The method in accordance with claim 15, wherein while in the Austenite phase, the nitinol wire is shape set to a curved, undulating, or spiral shape.

17. The method in accordance with claim 11, wherein the body is disposed within an outer sheath connected thereto via a living hinge acting as the stabilizing structural member isolating the distal tip of the body from the outer sheath so that the axial movement of the body while applying the aspiration is not transmittable to the outer sheath.

18. The method in accordance with claim 11, wherein the stabilizing structural member is a knurled outer surface of a portion of the body itself or an inflatable balloon disposed thereabout.

19. The method in accordance with claim 11, wherein the aspiration catheter further comprises an outer sleeve disposed about the body and secured together via an elastomeric polymer defining an air gap therebetween serving as the stabilizing structural member stabilizing the body minimizing movement of the distal tip when applying the aspiration; wherein the elastomeric polymer restores the body to a position substantially centered within the outer sleeve.

20. The method in accordance with claim 11, wherein the navigating step comprises the steps of:
advancing the aspiration catheter through an outer guide in the distal direction towards the targeted clot; and
engaging the targeted clot with the aspiration catheter while subject to aspiration and radially self-transitioning the radially enlargeable distal section from a radially contracted state to a radially expanded state;
wherein the stabilizing step comprises preventing the radial overexpansion of the distal tip of the body via a tether acting as the stabilizing structural member to minimize risk of damage to the inner wall of the vessel.
